(19) United States
(12) Patent Application Publication
(10) Pub. No.: US 2025/0336518 A1
(43) Pub. Date: Oct. 30, 2025

(54) REMOTE ASSISTANCE OF A MEDICAL INTERVENTION

(71) Applicant: Siemens Healthineers AG, Forchheim (DE)

(72) Inventor: Rebecca Fahrig, Mohrendorf (DE)

(21) Appl. No.: 19/177,169

(22) Filed: Apr. 11, 2025

(30) Foreign Application Priority Data
Apr. 24, 2024 (EP) 24172219

(51) Int. Cl. G16H 40/67; A61B 34/00; A61B 34/35; A61F 2/06

(52) U.S. Cl. CPC G16H 40/67; A61B 34/25; A61B 34/35; A61F 2/06; A61B 2034/258

(57) ABSTRACT

For remote assistance of a medical intervention, an input specifying a proposed medical device is received by a data processing system. Representation data for generating a visual representation of the medical device is generated by the data processing system depending on the input and transmitted to a remotely located display device. The visual representation is displayed by the display device depending on the representation data and a display size of the display device such that the displayed visual representation corresponds the proposed medical device in real size or a further visual representation of a reference object and the visual representation are displayed by the display device true to scale.

The present patent document claims the benefit of European Patent Application No. 24172219, filed Apr. 24, 2024, which is hereby incorporated by reference in its entirety.

### TECHNICAL FIELD

The present disclosure is directed to a method for remote assistance of a

medical intervention, to a system for carrying out such a method, and to a corresponding computer program product.

### BACKGROUND

Medical interventions, (e.g., interventions on vessels including, but not limited to, cardiovascular interventions or interventions on cerebral vessels), may be carried out by a first person and/or robot, also denoted as operator in the following, who is assisted by a remotely located second person, also denoted as remote expert in the following. This is sometimes also denoted as remote surgery or telesurgery. This has, for example, the advantage that a single remote expert may assist several interventions at the same time or shortly one after the other and does not have to be present at the location of the intervention.

For carrying out such interventions, the operator may select a medical device from a plurality of medical devices or modify a generic medical device. The operator may be instructed by the remote expert to do so via remote verbal communication or text-based communication or gestures delivered via video link. It may be difficult for the operator to correctly understand or implement the instructions as intended by the remote expert. As a consequence, the operator may select the wrong medical device or modify the generic medical device in a wrong way, and thus the process may be delayed.

### SUMMARY AND DESCRIPTION

It is an objective of the present disclosure to improve the remote assistance of a medical intervention such that the risk that the operator selects the wrong medical device or modifies the generic medical device in a wrong way is reduced.

The scope of the present disclosure is defined solely by the appended claims and is not affected to any degree by the statements within this summary. The present embodiments may obviate one or more of the drawbacks or limitations in the related art.

The disclosure is based on the idea to display a visual representation of a proposed medical device, which is proposed, in particular, by the remote expert to the operator, in real size or together with a reference object true to scale at the side of the operator.

According to an aspect, a method for remote assistance of a medical intervention is provided. Therein, an input, (e.g., a user input such as a user input of a remote expert), specifying a proposed medical device is received by a data processing system. Representation data for generating a visual representation of the proposed medical device is generated by the data processing system depending on the input. The representation data is transmitted from the data processing system to a display device, which is located remotely from the data processing system. The visual representation is displayed by the display device depending on the representation data and depending on a display size of the display device such that the displayed visual representation corresponds to the proposed medical device in real size. Alternatively, a further visual representation of a reference object and the visual representation are both displayed by the display device true to scale.

It is noted that the method does not contain acts for carrying out the medical intervention, in particular by using the selected or modified medical device.

The input may be received by the data processing system via a user input interface including, for example, a speech input interface and/or a text input interface, for example, from the remote expert. The user input interface may also be part of the data processing system.

For example, a medical device may be selected from a plurality of medical devices according to the displayed visual representation or a generic medical device may be modified according to the displayed visual representation. The selection or modification may be carried out manually, automatically, or in part automatically.

The selection of the device or the modification of the device may be carried out by an operator or partly by the operator. The operator and the display device are located at the same location and remotely from the data processing system and, in particular, the remote expert. The operator and the display device are located, in particular, in an intervention room in which the medical intervention shall be carried out. The data processing system and the display device being located remotely from each other may be understood that the user input interface and, for example, the remote expert are located outside of the intervention room. The user input interface and the intervention room may be located far away from each other in the sense that it is not possible for the remote expert and the operator to communicate with each other directly verbally or to see each other directly.

The display device may be part of a further data processing system or may be connected to the further data processing system. Transmitting the representation data from the data processing system to the display device may therefore be realized by transmitting the representation data from the data processing system to the further data processing system and the further data processing system may control the display device to display the visual representation. In case the display device possesses a respective interface, it may also be possible that the representation data is transmitted from the data processing system directly to the display device.

The display device may include a two-dimensional array of pixels for displaying the visual representation. The array of pixels may have a width of W pixels and a height of H pixels. Consequently, a total number of pixels of the array of pixels is W×H. The display size may be given by W and H or, in other words, the total number of pixels W×H and the aspect ratio of the array of pixels, and a pixel size of the individual pixels of the array. These data may be stored in a memory of the display device, a memory of the data processing system, and/or a memory of the further data processing system.

The display size may be received by the further data processing system. The further data processing system may then adapt the representation data accordingly depending on the display size such that the displayed visual representation corresponds to the proposed medical device in real size. Alternatively, the display size may be received by the data processing system and the data processing system may generate the representation data depending on the display size such that the further data processing system may control the display device accordingly to display the proposed medical device in real size.

The transmission of the representation data from the data processing system to the display device and/or to the further data processing system may include a transmission via a local area network (LAN), a wide area network (WAN), the internet, an ethernet connection, a Wi-Fi connection, a Bluetooth connection, and/or a cellular network.

The display device may be any type of device capable of displaying the visual representation based on the transmitted representation data, such as a computer screen, a screen of a television, a smartphone, or a tablet computer, a head-up-display device, a head-mounted display device, augmented reality glasses, virtual reality glasses, and so forth.

Some but not all of these types of display devices may be able to display the visual representation such that it corresponds to the proposed medical device in real size, which may be understood such that, if the real physical medical device would be held next to the displayed visual representation, both would have the same size and shape and proportions. This may be possible with a screen as the display device. With other types of display devices, such as a head-up-display device or augmented reality glasses, for example, this may not be possible or feasible. With such devices, it is possible to display the visual representation together with the further visual representation of the reference object, which may be an anatomic object or a further medical device, true to scale, for example, next to each other or one of them overlayed over the other, et cetera. Displaying the visual representation together with the further visual representation true to scale is also possible with a screen as the display device.

The representation data may be any data, which is generated based on the input, which allows the display device to directly display the visual representation and, in some implementations, the further visual representation or which allows the further data processing system to control the display device to display the visual representation and, in some implementations, the further visual representation.

The medical device may be any medical device to be used during the medical intervention including, but not limited to, tools, implants, and auxiliary devices. In particular, the medical device may be a catheter, (e.g., a vessel catheter), a stent, a vessel prosthesis, or a guidewire for guiding a further medical device, such as a catheter or stent, in a vessel structure.

By the method, the remote assistance of the operator by the remote expert to carry out the medical intervention is improved, because the risk that the operator selects the wrong medical device or modifies the generic medical device in a wrong way is reduced. This is achieved, in particular, by displaying the visual representation in real size or true to scale with respect to the reference object. Thus, the operator may directly verify their selection of the medical device or modification of the generic medical device based on the displayed visual representation.

In the present disclosure, the expressions “data processing system” and “at least one data processing device” may be used interchangeably. A data processing device may be understood as a data processing device, which includes processing circuitry. The data processing device may process data to perform computing operations. This may also include operations to perform indexed accesses to a data structure, for example, a look-up table (LUT), as well as a data processing process implemented in hardware.

In particular, the data processing device may include one or more computers, one or more microcontrollers, and/or one or more integrated circuits, for example, one or more application-specific integrated circuits (ASIC), one or more field-programmable gate arrays (FPGA), and/or one or more systems on a chip (SoC). The data processing device may also include one or more processors, for example, one or more microprocessors, one or more central processing units (CPU), one or more graphics processing units (GPU), and/or one or more signal processors, in particular one or more digital signal processors (DSP). The data processing device may also include a physical or a virtual cluster of computers or other of the units.

In various embodiments, the data processing device includes one or more hardware and/or software interfaces and/or one or more memory units.

A memory unit may be implemented as a volatile data memory, (e.g., a dynamic random access memory (DRAM) or a static random access memory (SRAM)), or as a non-volatile data memory, (e.g., a read-only memory (ROM), a programmable read-only memory (PROM), an erasable programmable read-only memory (EPROM), an electrically erasable programmable read-only memory (EEPROM), a flash memory or flash (EEPROM), a ferroelectric random access memory (FRAM), a magnetoresistive random access memory (MRAM), or a phase-change random access memory (PCRAM)).

According to several implementations, a size, (e.g., a geometrical size), and/or a shape property, (e.g., a property of a geometrical shape), of the proposed medical device is displayed in real size or true to scale, (e.g., true to scale with respect to the reference object), by displaying the visual representation. The size and/or shape property may be specified by the input.

Consequently, the visual representation of the medical device provides an accurate portrayal of its dimensions and shape, allowing to accurately assess the proposed medical device's size and shape before making the selection or modification. In such implementations, an added level of precision and accuracy in the selection or modification of the medical device may be achieved. Displaying the size or shape in real size or true to scale may help to clarify uncertainties regarding the medical device, making the selection or modification process more efficient.

The size of the proposed medical device may correspond to dimensions of the proposed medical device or a specific part of the medical device, which may be expressed in terms of length, width, height, diameter, thickness, and so forth. In other words, the size may be understood as a measurable characteristic of the proposed medical device or the specific part of the medical device, representing the extent or magnitude of its dimensions.

The shape property of the proposed medical device may refer to a characteristic of the proposed medical device that defines its geometric shape or form. This may include properties such as curvature, angles, contour, and/or other geometric features. In case the proposed medical device is or includes a guide wire, the shape property may correspond to a curvature of a curved tip or an angle of a tilted tip of the guide wire et cetera.

According to several implementations, a medical device is selected from a plurality of medical devices according to the displayed visual representation or a generic medical device is modified according to the displayed visual representation. Sensor data representing the selected or modified medical device is generated by a sensor system. The selection or modification of the medical device is validated depending on the sensor data.

The sensor data may include information about the size, shape, or other physical characteristics of the selected or modified medical device, which may be compared to the proposed medical device. The validation of the selection or modification of the medical device depending on the sensor data may include confirming or rejecting the selection or modification of the medical device. If it is confirmed, the operator may proceed using the selected or modified medical device and otherwise, another selection, and/or modification may be required. This adds an additional level of precision and accuracy to the remote assistance method.

The sensor system may include one or more imaging sensors, such as a camera, a stereo camera, a 3D camera, (e.g., a time-of-flight camera), and so forth. These may also be used to capture images of the selected or modified medical device from different angles. The sensor system may include one or more other optical sensors, such as laser scanners or optical probes, which may measure the dimensions and shape of the selected or modified medical device with particularly high precision. The sensor system may also include a touch-sensitive device, which may directly interact with the selected or modified medical device by touching the touch-sensitive device with the selected or modified medical device (“device-to-touch”).

According to several implementations, the sensor data is transmitted to the data processing system, in particular by the sensor system. A validation output, in particular for the remote expert, is generated by the data processing system depending on the sensor data. The selection or modification of the medical device is validated in response to the validation output.

Consequently, it may be provided with increased reliability that the selected or modified medical device is appropriate for the medical intervention. For example, the validation output may be a visual and/or auditory output that allows the remote expert to decide whether the selected or modified medical device is correctly selected or modified or not. The validation output may include the sensor data or a visual and/or auditory representation of the sensor data or a part of it. That the selection or modification of the medical device is validated in response to the validation output implies, in particular, that the validation is done after the validation output has been generated.

According to several implementations, a further input confirming or rejecting the selection or modification of the medical device is received by the data processing system, for example, via the user input interface, in particular from the remote expert, in response to the validation output. A confirmation message is transmitted from the data processing system to the display device or to a further output device if the further input confirms the selection or modification of the medical device. The further output device is, in particular, located remotely from the data processing system. The further output device may be located in the intervention room.

If the confirmation message is received, for example, by the operator, the operator may proceed further with using the selected or modified medical device for the medical intervention.

On the other hand, if the further input does not confirm, in particular does reject, the selection or modification of the medical device, a rejection message may be transmitted from the data processing system to the display device or to the further output device. The operator may then select another medical device or adapt the modification of the generic medical device or the acts of the method may be repeated to find the correct proposed medical device.

The further input confirming or rejecting the selection or modification may, in some implementations also be generated automatically, for example, by an AI-based algorithms. The confirmation message or the rejection message may also be transmitted to the remote expert. The remote expert may also be prompted to suggest alternatives or changes and so forth.

According to several implementations, for generating the sensor data, the size, and/or the shape property of the selected or modified medical device is measured by the sensor system. The selection or modification of the medical device is validated depending on the measured size and/or the shape property. In particular, the sensor data includes the measured size and/or the shape property.

Consequently, a particularly reliable validation loop is established. The measurement may be carried out by the further data processing system and/or by a data processing device of the sensor system. For example, in case the sensor system is a laser scanner or optical probe, the sensor data may already include the respective measurement data. In case the sensor system is a camera, the measurement may include a computer program for measuring the size and/or the shape property being applied to the respective image or images. The computer program may include an object detection algorithm, an image segmentation algorithm, et cetera.

According to several implementations, for generating the sensor data, a camera image, or a video of the selected or modified medical device is generated by at least one camera, in particular by at least one camera of the sensor system. The selection or modification of the medical device is validated depending on the camera image or the video. In particular, the sensor data includes the camera image or the video.

According to several implementations, for generating the sensor data, a three-dimensional scan of the selected or modified medical device is generated by a laser scanner, in particular a laser scanner of the sensor system. The selection or modification of the medical device is validated depending on the three-dimensional scan.

This allows for a particularly precise characterization of the selected or modified medical device and thus for a particularly reliable validation. In particular, the sensor data includes the three-dimensional scan.

According to several implementations, further sensor data representing the proposed medical device is generated by a further sensor system and the input includes the further sensor data or is generated based on the further sensor data.

In other words, the user input is generated based on a real physical version of the proposed medical device. Consequently, the reliability of the process is further improved. For example, the remote expert may have the proposed medical device at hand and use the further sensor system to generate the input. The explanations and examples above regarding the sensor system apply analogously to the further sensor system.

According to a further aspect, a system for carrying out a method for remote assistance of a medical intervention is provided. The system includes the data processing system and the display device.

In some implementations, the system also includes the sensor system and/or the further sensor system.

According to several implementations, the system includes a robotic manipulator, which is configured to select the medical device from the plurality of medical devices according to the displayed visual representation and/or to modify the generic medical device according to the displayed visual representation.

The robotic manipulator may select the medical device or modify the generic medical device automatically, for example, autonomously, or may be controlled by a human operator to do so.

Above and in the following, the solution is described with respect to the claimed systems as well as with respect to the claimed methods. Features, advantages, or alternative embodiments herein may be assigned to the other claimed objects and vice versa. In other words, claims and embodiments for the systems may be improved with features described or claimed in the context of the respective methods. In this case, the functional features of the method are implemented by physical units of the system.

According to a further aspect, a computer program including instructions is provided. When the instructions are executed by a system, in particular by its data processing system, the instructions cause the data processing system to receive the input, generate the representation data, and transmit the representation data to the display device, and cause the display device to display the visual representation.

The instructions may be provided as program code, for example. The program code may be provided as binary code or assembler and/or as source code of a programming language, (e.g., C), and/or as program script, (e.g., Python).

According to several implementations, the instructions, when executed by the system, in particular by its data processing system, cause the sensor system to generate sensor data and/or cause the sensor system to transmit the sensor data to the data processing system and/or cause the data processing system to generate a validation output depending on the sensor data.

Further implementations of the computer program follow directly from the various embodiments of the method and vice versa. In particular, individual features and corresponding explanations as well as advantages relating to the various implementations of the method may be transferred analogously to corresponding implementations of the computer program.

According to a further aspect, a computer-readable storage medium storing a computer program is provided.

The computer program and the computer-readable storage medium are respective computer program products including the instructions.

Further features and feature combinations of the disclosure are obtained from the figures and their description as well as the claims. In particular, further implementations of the disclosure may not necessarily contain all features of one of the claims. Further implementations of the disclosure may include features or combinations of features, which are not recited in the claims.

In the following, the disclosure is explained in detail with reference to specific exemplary implementations and respective schematic drawings. In the drawings, identical or functionally identical elements may be denoted by the same reference signs. The description of identical or functionally identical elements is not necessarily repeated with respect to different figures.

### BRIEF DESCRIPTION OF THE DRAWINGS

FIG. 1 depicts a schematic representation of an exemplary implementation of a system.

FIG. 2 depicts a schematic representation of a further exemplary implementation of a system.

FIG. 3 depicts a flow diagram of an exemplary implementation of a method for remote assistance of a medical intervention.

FIG. 4 depicts a flow diagram of a further exemplary implementation of a method for remote assistance of a medical intervention.

FIG. 5 depicts a flow diagram of a part of a further exemplary implementation of a method for remote assistance of a medical intervention.

### DETAILED DESCRIPTION

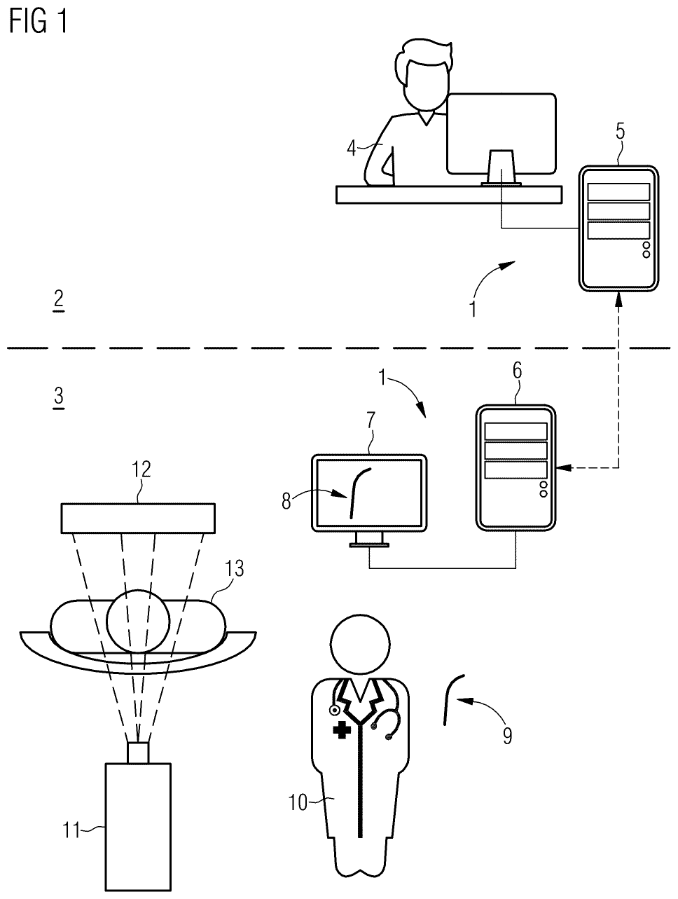

FIG. 1 shows a schematic representation of an exemplary implementation of a system 1 for remote assistance of a medical intervention.

The system 1 includes a data processing system 5 for use by a remote expert 4. The system 1 further includes a display device 7, which is located remotely from the data processing system 5 and the remote expert 4. The display device 7 is coupled to the data processing system 5, for example via a further data processing system 6, which is also located remotely to the data processing system 5. The display device 7 may display information to an operator 10, who may carry out the medical intervention. In particular, the remote expert 4 may communicate with the operator 10 via the data processing system 5, optionally the further data processing system 6, and the display device 7. In this way, the remote expert 4 may assist and/or instruct the operator 10 when carrying out the medical intervention.

The operator 10 and the display device 7 are located at the same location and remotely from the data processing system 5 and, in particular, the remote expert 4. The operator 10 and the display device 7 are, for example, located in an intervention room 3 in which the medical intervention shall be carried out. The data processing system 5 and the remote expert 4 are located outside of the intervention room, for example in a control room 2.

In a non-limiting example, the medical intervention may be a minimally-invasive intervention concerning a vessel structure of a patient 13 with fluoroscopic assistance by using an x-ray imaging system including an x-ray source 11 and an x-ray detector 12.

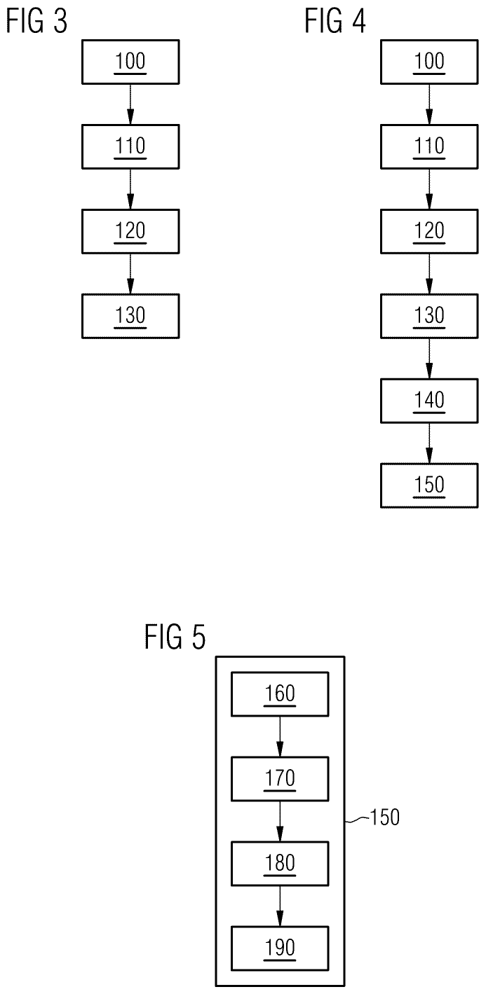

The system 1 is configured to carry out a method for remote assistance of a medical intervention. A corresponding flow diagram is shown in FIG. 3.

In act 100, the data processing system 5 receives an input specifying a proposed medical device, in particular from the remote expert 4 via a user input interface of the data processing system 5.

In act 110, the data processing system 5 generates representation data for generating a visual representation 8 of the proposed medical device depending on the received input and transmits it to the display device 7, for example, via the further data processing system 6.

In act 120, the visual representation 8 is displayed by the display device 7 depending on the representation data and depending on a display size of the display device 7 such that the displayed visual representation 8 corresponds to the proposed medical device in real size or a further visual representation of a reference object and the visual representation 8 are displayed by the display device 7 true to scale.

In optional act 130, a medical device is selected from a plurality of medical devices according to the displayed visual representation 8 or a generic medical device is modified according to the displayed visual representation, for example by the operator 10.

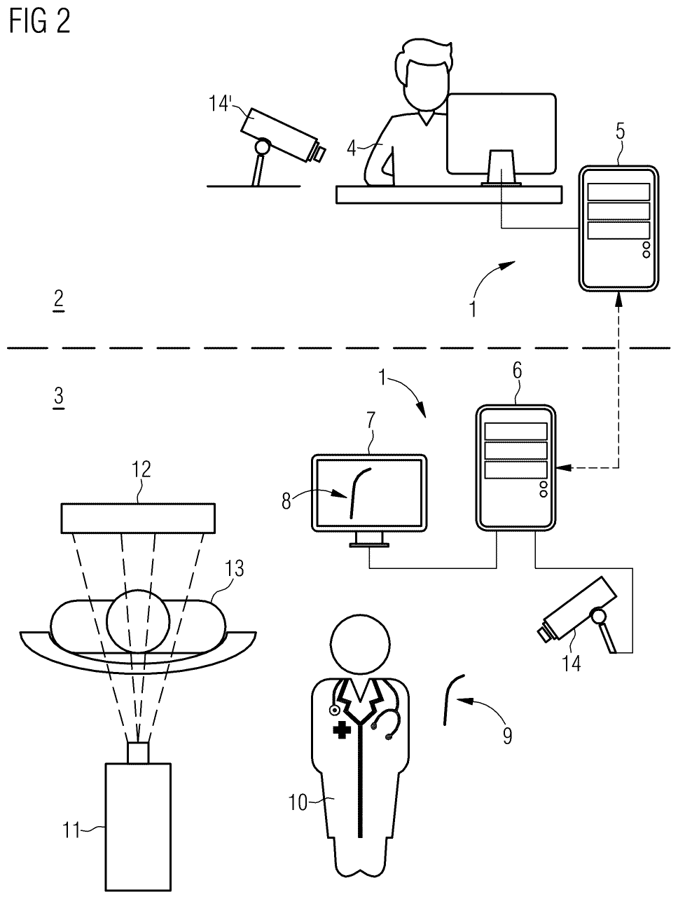

FIG. 2 shows a schematic representation of a further exemplary implementation of a system 1 for remote assistance of a medical intervention, which is based on the implementation of FIG. 1.

Therein, the system 1 includes a sensor system 14, such as a camera or a laser scanner or the like. The sensor system 14 may generate sensor data, for example, one or more camera images or a video or a three-dimensional scan, and provide it, for example, via the further data processing system 6 to the data processing system 5. FIG. 3 shows a flow diagram of further exemplary implementation of a method for remote assistance of a medical intervention, which may be carried out using the system 1 of FIG. 2, and which is based on the method of FIG. 3. Acts 100 to 130 of the method according to FIG. 4 are identical to the acts 100 to 130 of the method according to FIG. 3.

In act 140, sensor data representing the selected or modified medical device 9 is generated by the sensor system 14.

In act 150, the selection or modification of the medical device is validated depending on the sensor data, for example, by the remote expert 4.

FIG. 5 shows a flow diagram of method acts of a further exemplary implementation of a method for remote assistance of a medical intervention, included within act 150 of the method of FIG. 4.

Therein, in act 160, the sensor data is transmitted from the sensor system 140 to the data processing system 5, for example, via the further data processing system 5.

In act 170, a validation output is generated by the data processing system 5 depending on the sensor data.

In act 180, a further input confirming or rejecting the selection or modification of the medical device is received by the data processing system 5, in particular from the remote expert 4 via the user input interface of the data processing system 5, in response to the validation output.

In act 190, a confirmation message is transmitted from the data processing system 5 to the display device 7 if the further input confirms the selection or modification of the medical device. The operator may then carry out the medical intervention using the selected or modified medical device 9.

As described, the disclosure improves the remote assistance of a medical intervention such that the risk that the operator 10 selects the wrong medical device or modifies the generic medical device in a wrong way is reduced.

For example, in a vessel intervention, when the remote expert 4 picks the appropriate medical devices for the next act in the intervention procedure, they may determine that a particular shape or curvature needs to be added to the medical device in order to navigate through the vessels efficiently and effectively. The desired shape may be shown directly on the display device 7, which may be a computer screen, a touchpad screen, or on a heads-up display, in real size or true to scale. Thus, a template is effectively provided to the operator 10, against which the medical device to be selected or modified may be compared directly.

In some implementations, direct feedback to the remote expert 4 may be provided via an optical camera, via device-to-touch screen interaction or via 3D scanning of the selected or modified medical device 9, in order to determine if the shape as provided by the operator 10 matches the proposed template. The direct feedback improves the remote expert's 4 confidence that the operator 10 has chosen the correct medical device and/or has implemented the correct shape modifications.

In some implementations, a 3D vision overlay may be used for the comparison of the proposed medical device against the true medical device.

In some implementations, an automated template selection may be provided. A prior imaging database that allows to determine, suggest and/or automatically calculate the best shape modification and/or device selection based on knowledge of a 3D vessel geometry of the patient 13 from prior 3D imaging technologies including, for example, computed tomography, magnetic resonance imaging, positron emission tomography, tomosynthesis, cone-beam computed tomography, and/or based on single-view angiography, two-view angiography, standard vessel geometries based on population statistics, et cetera. Thus, proposed medical device or shape itself is provided automatically.

In some implementations, the remote expert 4 may have the exact medical devices in hand and/or modify a generic medical device, and then, for example, through a further sensor device 14′, such as a touch-display and/or an optical scanning device or a camera, the proposed medical device may be visualized to the operator 10.

It is to be understood that the elements and features recited in the appended claims may be combined in different ways to produce new claims that likewise fall within the scope of the present disclosure. Thus, whereas the dependent claims appended below depend on only a single independent or dependent claim, it is to be understood that these dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for remote assistance of a medical intervention, the method comprising:
receiving, by a data processing system, an input specifying a proposed medical device;
generating, by the data processing system, representation data depending on the input; and
transmitting the representation data by the data processing system to a display device located remotely from the data processing system; and
displaying, by the display device, (1) a visual representation depending on the representation data and depending on a display size of the display device such that a size of the displayed visual representation corresponds to a real size of the proposed medical device, or (2) a further visual representation of a reference object and the visual representation true to scale.

2. The method of claim 1, wherein the reference object is an anatomic object or a further medical device.

3. The method of claim 1, wherein a size and/or a shape property of the proposed medical device is displayed in real size or true to scale by displaying the visual representation.

4. The method of claim 1, wherein a medical device is selected from a plurality of medical devices according to the displayed visual representation or a generic medical device is modified according to the displayed visual representation,
wherein sensor data representing the selected or modified medical device is generated by a sensor system, and
the selection or modification of the medical device is validated depending on the sensor data.

5. The method of claim 4, wherein the sensor data is transmitted to the data processing system,
wherein a validation output is generated by the data processing system depending on the sensor data, and
wherein the selection or modification of the medical device is validated in response to the validation output.

6. The method of claim 5, wherein a further input confirming or rejecting the selection or modification of the medical device is received by the data processing system in response to the validation output, and
wherein a confirmation message is transmitted from the data processing system to the display device or to a further output device when the further input confirms the selection or modification of the medical device.

7. The method of claim 4, wherein a size and/or a shape property of the proposed medical device is displayed in real size or true to scale by displaying the visual representation,
wherein the size and/or the shape property of the selected or modified medical device is measured by the sensor system for the generating of the sensor data, and
wherein the selection or modification of the medical device is validated depending on the size and/or the shape property.

8. The method of claim 4, wherein a size and/or a shape property of the proposed medical device is displayed in real size or true to scale by displaying the visual representation,
wherein a camera image or a video of the selected or modified medical device is generated by at least one camera for the generating of the sensor data, and
wherein the selection or modification of the medical device is validated depending on the camera image or the video.

9. The method of claim 4, wherein a size and/or a shape property of the proposed medical device is displayed in real size or true to scale by displaying the visual representation,
wherein a three-dimensional scan of the selected or modified medical device is generated by a laser scanner for the generating of the sensor data, and
wherein the selection or modification of the medical device is validated depending on the three-dimensional scan.

10. The method of claim 1, further comprising:
generating further sensor data representing the proposed medical device by a further sensor system, and
wherein the input comprises the further sensor data or is generated based on the further sensor data.

11. The method of claim 1, wherein the proposed medical device is a stent, a vessel prosthesis, a vessel catheter, or a guidewire for guiding a further medical device in a vessel structure.

12. A system comprising:
a data processing system; and
a display device located remotely from the data processing system,
wherein the data processing system is configured to:
receive an input specifying a proposed medical device;
generate representation data depending on the input; and
transmit the representation data to the display device,
wherein the display device is configured to:
(1) display a visual representation depending on the representation data and depending on a display size of the display device such that a size of the displayed visual representation corresponds to a real size of the proposed medical device; or
(2) display a further visual representation of a reference object and the visual representation true to scale.

13. The system of claim 12, further comprising:
a robotic manipulator configured to select a medical device from a plurality of medical devices according to the displayed visual representation or to modify a generic medical device according to the displayed visual representation.

14. A computer program product comprising instructions, which, when executed by a system, cause the system to:
receive, by a data processing system of the system, an input specifying a proposed medical device;
generate, by the data processing system, representation data depending on the input;
transmit the representation data by the data processing system to a display device of the system located remotely from the data processing system; and
display, by the display device, (1) a visual representation depending on the representation data and depending on a display size of the display device such that a size of the displayed visual representation corresponds to a real size of the proposed medical device, or (2) a further visual representation of a reference object and the visual representation true to scale.
